(19) United States
(12) Patent Application Publication
(10) Pub. No.: US 2021/0212571 A1
(43) Pub. Date: Jul. 15, 2021

(54) INTRAVASCULAR PHOTOACOUSTIC TOMOGRAPHY APPARATUS AND METHOD THEREOF

(71) Applicants: Ji-xin Cheng, Newton, MA (US); Yingchun Cao, Natick, MA (US); Purdue Research Foundation, West Lafayette, IN (US)

(72) Inventors: Ji-xin Cheng, Newton, MA (US); Yingchun Cao, Natick, MA (US)

(21) Appl. No.: 17/055,260

(22) PCT Filed: May 16, 2019
(86) PCT No.: PCT/US19/32673

§ 371 Date: Nov. 13, 2020

(51) Int. Cl. A61B 5/00; A61B 5/02

(52) U.S. Cl. CPC A61B 5/0095; A61B 5/0084; A61B 5/02007; A61B 5/0073; A61B 5/6852

(57) ABSTRACT

An apparatus and method for converting localized laser absorption in lipid-rich biological tissue into ultrasonic waves through thermoelastic expansion to image the entire arterial wall with chemical selectivity and depth resolution. The apparatus including a sensitive quasi-collinear dual-mode photoacoustic/ultrasound catheter with elaborately selected sheath material.

### CROSS-REFERENCE TO RELATED APPLICATION

This Patent Application claims priority to U.S. Provisional Application: 62/672318 filed May 16, 2018, the disclosure of which is considered part of the disclosure of this application and is hereby incorporated by reference in its entirety.

### GOVERNMENT RIGHTS

This invention was made with government support under NIH-HL125385 awarded by the National Institutes of Health. The government has certain rights in the invention.

### FIELD OF THE INVENTION

This invention relates generally to photoacoustic tomography, and more specifically to intravascular photoacoustic tomography for assessment of lipid content in arteries.

### BACKGROUND

Coronary artery disease is the leading cause of mortality worldwide. The disease refers to the pathologic development of atheromatous plaques in the coronary arterial tree and the subsequent narrowing of the lumen or even formation of thrombus due to plaque rupture, leading to restriction of blood flow and life-threatening acute coronary syndrome. Plaques that are considered most susceptible to rupture, or vulnerable plaques, are those with a large lipid-rich necrotic core, covered by a thin fibrous cap, and dense inflammatory infiltrate. Reliable and accurate detection of vulnerable plaques would ideally include not only morphological information of the artery wall, but also chemical composition of the suspected lesion. Intravascular ultrasound (IVUS) and optical coherence tomography can provide important morphological information of an artery. However, they lack chemical selectivity to accurately assess plaque composition. Near-infrared spectroscopy combined with IVUS has been shown to detect the presence of lipid-rich plaques and quantify them with a lipid core burden index, yet lack depth resolution to quantify and localize the cholesterol accumulation in lipid-rich plaques.

Intravascular photoacoustic (IVPA) tomography is an emerging catheter-based technology for the localization, quantification, and characterization of lipid deposition while simultaneously complementing traditional IVUS. The biggest advantage is that it can provide lipid-specific detection with depth resolution cover the entire arterial wall by converting light absorption into ultrasound (US) detection. Over the past several years, efforts have been made towards technical improvement of IVPA technique to meet clinical requirements including the report of various catheter designs, the development of laser sources for increased lipid sensitivity and imaging speeds, and differentiation of multiple tissue components. Nevertheless, catheter sensitivity in current designs has been the greatest obstacle for in vivo demonstration. Front-and-back designs exhibit an insufficient depth range to encompass the entire artery wall; co-axial designs are limited by transducer dimensions making the catheter too large for coronary artery access; Co-linear catheter designs have shown improved photoacoustic (PA) sensitivity and depth, but poor US resolution due to considerable signal loss at multiple reflective surfaces.

In addition, a proper protective sheath material that is transparent to both PA and US signals is essential for in vivo application but has yet to be identified. In vivo IVPA imaging has been previously attempted in animal models, however, incomplete technical preparations, such as lack of a protective sheath, lack of morphological feature provided by US, and artificial plaque, blood clearance and unsuitable sheath material, prevent them from functioning well and providing valuable information under clinically relevant conditions.

IVPA imaging can bring forth novel capabilities for the detection of lipid-rich atherosclerotic plaques and perivascular adipose tissue without displacement or occlusion of blood flow. It is increasingly accepted that atherosclerotic lesions primarily develop in arteries with perivascular. An imaging system is needed for the localization and quantification of lipid deposition across the entire arterial walls, including perivascular adipose tissue. adipose and surgical removal of the adipose encasing the arteries attenuates atherogenesis.

Currently, sensitivity remains the most important technical challenge for IVPA to be applied to in vivo study. Therefore, there exists a need for an apparatus and method to enable in vivo IVPA imaging of native arteries. The present disclosure presents an imaging apparatus and method having a quasi-collinear IVPA catheter with high sensitivity and sufficient depth and selected a sheath material with minimal PA and US attenuation and artifact generation. The method and apparatus of the present disclosure enables in vivo IVPA imaging of native arteries under clinically relevant conditions with real-time display up to about 16 frames per second (fps). The apparatus of method can be used for localization and quantification of lipid content along the full depth of the arterial wall from intima to perivascular adipose tissue for pullback lengths up to about 80 mm.

### BRIEF SUMMARY OF THE INVENTION

In one aspect, this disclosure is related to an intravascular photoacoustic tomography apparatus, comprising a light source configured to emit a light beam at a desired wavelength. A pulser receiver can be included and configured to send and receive the ultrasound pulses. Additionally, the apparatus can include delay generator configured to delay and trigger the ultrasound pulses from the pulser receiver. The apparatus can further include a processing means configured to control the pulser receiver, light source, and delay generator. A connector having a first end and second end can be used to couple the light source and the catheter. Additionally, a coupling means, such as a multimode fiber can be configured to communicatively couple the light source to the first end of the connector. The catheter can have a first end and a second end, wherein the catheter can be coupled to the second end of the connector. The catheter can further include an imaging probe portion, wherein said imaging probe portion comprises a mirror, transducer, and optical fiber. The catheter can be coupled directly or indirectly to the stage. The stage is configured to move along at least a first axis.

In another aspect, this disclosure is related to a method for imaging an artery wall for lipid deposits. The method can include providing an IVPA apparatus comprising a light source, a delay generator, a puller/receiver, a digitizer, processing means/computer, a multimode fiber, a Luer-slip connector, a hybrid rotary join and motorized stage and a catheter. The catheter of the IVPA can first be inserted inside an artery having an artery wall. A light beam from the light source can then be directed to the arterial wall for lipid-specific excitation by a multimode fiber and imaging probe portion of the catheter. The artery wall can be photoacoustically stimulated with optical energy from the light beam directed by the imaging prober portion. The ultrasonic signals generated from said tissue via the transducer array can be captured and transmitted to the processing means. The focus spot can be repositioned within the artery wall by pulling back the catheter through the artery a pre-determined distance and direction and repeating the steps of stimulating the artery wall and capturing ultrasonic signals. The processing means can then process the signals and generate an image of the tissue by combining the captured photoacoustic and ultrasonic signals from the various scans conducted by the IVPA at various positions within the artery.

The invention now will be described more fully hereinafter with reference to the accompanying drawings, which are intended to be read in conjunction with both this summary, the detailed description and any preferred and/or particular embodiments specifically discussed or otherwise disclosed. This invention may, however, be embodied in many different forms and should not be construed as limited to the embodiments set forth herein; rather, these embodiments are provided by way of illustration only and so that this disclosure will be thorough, complete and will fully convey the full scope of the invention to those skilled in the art.

### BRIEF DESCRIPTION OF THE DRAWINGS

The features and advantages of this disclosure, and the manner of attaining them, will be more apparent and better understood by reference to the following descriptions of the disclosed system and process, taken in conjunction with the accompanying drawings, wherein:

FIG. 1A is a diagram of an exemplary embodiment of an intravascular photoacoustic (IVPA) imaging system of the present disclosure.

FIG. 1B is a schematic of an exemplary embodiment of an intravascular photoacoustic (IVPA) imaging system of the present disclosure.

FIG. 1C is a graph illustrating the absorption coefficient of lipids and water using the imaging system of the present disclosure.

FIG. 1D is an illustration of an exemplary embodiment of an IVPA imaging system further illustrating the imaging probe portion.

FIG. 1E is an enlarged view of the imaging probe portion of FIG. 1D.

FIG. 2A is a photoacoustic/ultrasound image of a bare catheter without a sheath.

FIG. 2B is a photoacoustic/ultrasound image of a catheter having a sheathing made from a fluorinated ethylene propylene (FEP) material.

FIG. 2C is a photoacoustic/ultrasound image of a catheter having a sheathing made from a polytetrafluoroethylene (PTFE) material.

FIG. 2D is a photoacoustic/ultrasound image of a catheter having a sheathing made from a polyimide (PI) material.

FIG. 2E is a photoacoustic/ultrasound image of a catheter having a sheathing made from polyethylene (PE) material.

FIG. 2F is a photoacoustic/ultrasound image of a catheter having a sheathing made from polyurethane (PU) material.

FIG. 3A is a diagram plan for in vivo IVPA imaging of rabbit aorta with a pullback length of 80 mm

FIG. 3B is an image of the IVPA catheter using a 6 Fr introducer sheath to access the left femoral artery for catheterization.

FIG. 3C is an image of the aorta that was excised for histology.

FIG. 4A is a diagram of a trigger signal generated by the excitation laser source and synchronized with optical pulses, ultrasound pulses with double frequency and about a 5-μs delay to optical pulses were sent by ultrasound puller/receiver to generate co-registered and definition-improved IVUS image for high-speed real-time imaging of the present disclosure.

FIG. 4B is an image of A-lines for both PA and US channels after bandpass filtering, Hilbert transform, and noise removal.

FIG. 4C is a Cartesian coordinate expression of PA and US images with designated pixel density of 90 pixel/mm and scale bar of 1 mm.

FIG. 4D are images of 3-dimensional (3D) PA and US images reconstructed from cross-sectional image stacks with merged display.

FIG. 5A is a cross-sectional photoacoustic image was reconstructed from the raw data obtained by the apparatus and method of present invention.

FIG. 5B-C are graphical illustrations of peak amplitude of photoacoustical signal along with radial direction detected and the corresponding depth that was recorded for each frame.

FIG. 5D-E are 2-dimensional images expressing the peak amplitude of photoacoustic signal and depth for the entire pullback to indicate lipid distribution and depth.

FIG. 5F is an image relating to FIG. 5A where a proper threshold (4 times of noise level in this work) was applied to photoacoustic image to generate a cross-sectional binary lipid map (i.e. 0 for background and 1 denotes lipid presence).

FIG. 5G is a graphical illustration of lipid presence along angular direction at a specific depth was plotted to the show the angle of view for lipid pools.

FIG. 5H is a graphical illustration of the angular ratio of the largest lipid pool, i.e. angle of view over 2π n in percentage, was generated for each depth.

FIG. 5I is a map image of angular ratio of largest lipid pool was produced along the longitudinal direction of the artery to provide a complementary information about the lipid pool size and distribution depth.

FIG. 5J is a graphical illustration of the total lipid area for each cross-section quantitate from FIG. 5f for the entire artery.

FIG. 6A is an image of a pullback of in vivo IVPA imaging of a rabbit aorta showing peak photoacoustic amplitude corresponding to rotational speed of about 4 fps and pull back speed of about 0.25 mm/s.

FIG. 6B is an image of a pullback of in vivo IVPA imaging of a rabbit aorta showing peak photoacoustic amplitude corresponding to rotational speed of about 16 fps and pull back speed of about 1 mm/s.

FIG. 6C is an image of a pullback of in vivo IVPA imaging of a rabbit aorta showing peak photoacoustic depth corresponding to rotational speed of about 4 fps and pull back speed of about 0.25 mm/s.

FIG. 6D is an image of a pullback of in vivo IVPA imaging of a rabbit aorta showing peak photoacoustic depth corresponding to rotational speed of about 16 fps and pull back speed of about 1 mm/s.

FIG. 7A is an image of a pullback of in vivo IVPA imaging of human right coronary artery (RCA) showing peak photoacoustic amplitude corresponding to rotational speed of about 4 fps and pull back speed of about 0.25 mm/s.

FIG. 7B is an image of a pullback of in vivo IVPA imaging of human right coronary artery (RCA)showing peak photoacoustic amplitude corresponding to rotational speed of about 16 fps and pull back speed of about 1 mm/s.

FIG. 7C is an image of a pullback of in vivo IVPA imaging of human right coronary artery (RCA) showing peak photoacoustic depth corresponding to rotational speed of about 4 fps and pull back speed of about 0.25 mm/s.

FIG. 7D is an image of a pullback of in vivo IVPA imaging of a human right coronary artery (RCA) showing peak photoacoustic depth corresponding to rotational speed of about 16 fps and pull back speed of about 1 mm/s.

FIG. 8A is an illustration of an exemplary design and evaluation of a quasi-collinear IVPA catheter of the imaging system of the present disclosure showing PA imaging depth ranging from 0.6 to >6 mm based on estimated divergence angles of 3° and 6° for ultrasound and optical beams, respectively.

FIG. 8B are combined PA images of a 7-μm carbon fiber at different distances from the catheter center from 1.4 to 4.6 mm. The insets showing the photo of the catheter tip and enlarged image of the target at a distance of 4.1 mm.

FIG. 8C is a graph for the PA axial resolution with an inset showing the PA signals across the target at an axial distance of about 4.1 mm along the axial direction.

FIG. 8D is a graph for the PA lateral resolution with an inset showing the PA signals across the target at an axial distance of about 4.1 mm along the lateral direction.

FIG. 8E is a graph illustrating the PA amplitude of the PA signals.

FIG. 9A is a graphical illustration of the performance of five different sheathing materials for IVPA imaging for the PA artifact measurement. The value of artifact is regarded as the maximum signal from the sheath.

FIG. 9B is a graphical illustration of the performance of five different sheathing materials for IVPA imaging for the PA transmission measurement. The transmission was determined by comparing with bare catheter situation.

FIG. 9C is a graphical illustration of the performance of five different sheathing materials for IVPA imaging for the US artifact measurement. The value of artifact is regarded as the maximum signal from the sheath.

FIG. 9D is a graphical illustration of the performance of five different sheathing materials for IVPA imaging for the US transmission measurement. The transmission was determined by comparing with bare catheter situation.

FIG. 9E is an IVPA image of a human coronary artery imaged ex vivo using a bare catheter without a sheath and with luminal PBS. The scale bar is 1 mm for cross-sectional images.

FIG. 9F is an IVPA image of a human coronary artery imaged ex vivo using a catheter with D2O-filled PU sheath and luminal PBS. The scale bar is 1 mm for cross-sectional images.

FIG. 9G is an IVPA image of a human coronary artery imaged ex vivo using a catheter with D2O-filled PU sheath and luminal blood. The scale bar is 1 mm for cross-sectional images.

FIG. 10A are in vivo IVPA imaging of a rabbit aorta. Labels I-III correspond to PA, merged PA/US images, and Verhoeff-van Gieson stained histopathology, respectively.

FIG. 10B are in vivo IVPA imaging of a rabbit aorta. Labels I-III correspond to PA, merged PA/US images, and Verhoeff-van Gieson stained histopathology, respectively.

FIG. 10C are in vivo IVPA imaging of a rabbit aorta. Labels I-III correspond to PA, merged PA/US images, and Verhoeff-van Gieson stained histopathology, respectively.

FIG. 10D is an x-ray angiogram image of an IVPA catheter in the thoracic aorta, with forceps and ruler to locate the position of the catheter externally.

FIG. 10E is a r reconstructed 3D merged PA/US image for a pullback segment of 20-mm length of the aorta. Images in this figure were collected at 4 fps and a pullback speed of 0.25 mm/s.

FIG. 11A is graphical representation of lipid core in rabbit aortas in vivo at the lipid core depth at each frame along a pullback length of 60 mm with different rotational and pullback speeds (4 fps and 0.25 mm/s vs. 16 fps and 1 mm/s). Lipid core depth corresponds to the depth to catheter center where PA signal shows a maximum amplitude.

FIG. 11B is graphical representation of lipid core in rabbit aortas in vivo at the lipid core angle at each frame along a pullback length of 60 mm with different rotational and pullback speeds (4 fps and 0.25 mm/s vs. 16 fps and 1 mm/s). Angle of lipid core means the observation angle of the maximum lipid core from catheter center.

FIG. 11C is graphical representation of lipid core in rabbit aortas in vivo at the lipid core area of lipids at each frame along a pullback length of 60 mm with different rotational and pullback speeds (4 fps and 0.25 mm/s vs. 16 fps and 1 mm/s). Area of lipids is obtained by counting all the lipids in and surrounding the arterial wall.

FIG. 11D is a graphical illustration of the average lipid core depth for two different rabbit aortas. The error bars are resulted from all the frames during entire pullbacks.

FIG. 11E is a graphical illustration of the average lipid core angle for two different rabbit aortas. The error bars are resulted from all the frames during entire pullbacks.

FIG. 11F is a graphical illustration of the average volume of lipids in a 1 mm artery length for two different rabbit aortas. The error bars are resulted from all the frames during entire pullbacks.

FIG. 12A is an ex vivo IVPA cross-sectional PA image of a human right coronary artery.

FIG. 12B is an ex vivo IVPA cross-sectional US image of a human right coronary artery.

FIG. 12C is an ex vivo IVPA cross-sectional merged PA/US image of a human right coronary artery.

FIG. 12D is a corresponding ex vivo the Movat's pentacrhome stained histopathology section image of a human right coronary artery of FIG. 12A.

FIG. 12E is an ex vivo IVPA cross-sectional PA image of a human right coronary artery.

FIG. 12F is an ex vivo IVPA cross-sectional US image of a human right coronary artery.

FIG. 12G is an ex vivo IVPA cross-sectional merged PA/US image of a human right coronary artery. The boundaries of lumen and external elastic membrane are outlined by dashed lines, respectively, to illustrate the intimal thickening observed on US image.

FIG. 12H is a corresponding ex vivo the Movat's pentacrhome stained histopathology section image of a human right coronary artery of FIG. 12E. Intimal thickening having lipid is shown by the arrows.

FIG. 12I illustrates the maximum PA amplitude at each radial direction (ϕ) from 0 to 360° along pullback direction (z) from 0 to 40 mm

FIG. 12J illustrates the corresponding depth from the center of the catheter.

FIG. 12K illustrates the angular ratio of maximum lipid pool at individual depth along the artery.

FIG. 12L illustrates the quantitated lipid area at each cross-section of the artery for the 40-mm pullback.

### DETAILED DESCRIPTION OF THE INVENTION

The following description includes discussion of figures having illustrations given by way of example of implementations of embodiments of the invention. The drawings should be understood by way of example, and not by way of limitation. As used herein, references to one or more “embodiments” are to be understood as describing a particular feature, structure, or characteristic included in at least one implementation of the invention. Thus, phrases such as “in one embodiment” or “in an alternate embodiment” appearing herein describe various embodiments and implementations of the invention, and do not necessarily all refer to the same embodiment. However, they are also not necessarily mutually exclusive.

Before the present invention of this disclosure is described in such detail, however, it is to be understood that this invention is not limited to particular variations set forth and may, of course, vary. Various changes may be made to the invention described and equivalents may be substituted without departing from the true spirit and scope of the invention. In addition, many modifications may be made to adapt a particular situation, material, composition of matter, process, process act(s) or step(s), to the objective(s), spirit or scope of the present invention. All such modifications are intended to be within the scope of the disclosure made herein.

Unless otherwise indicated, the words and phrases presented in this document have their ordinary meanings to one of skill in the art. Such ordinary meanings can be obtained by reference to their use in the art and by reference to general and scientific dictionaries.

References in the specification to “one embodiment” indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The following explanations of certain terms are meant to be illustrative rather than exhaustive. These terms have their ordinary meanings given by usage in the art and in addition include the following explanations.

As used herein, the term “and/or” refers to any one of the items, any combination of the items, or all of the items with which this term is associated.

As used herein, the singular forms “a,” “an,” and “the” include plural reference unless the context clearly dictates otherwise.

As used herein, the terms “include,” “for example,” “such as,” and the like are used illustratively and are not intended to limit the present invention.

As used herein, the term “coupled” means the joining of two members directly or indirectly to one another. Such joining may be stationary in nature or movable in nature. Such joining may be achieved with the two members or the two members and any additional intermediate members being integrally formed as a single unitary body with one another or with the two members or the two members and any additional intermediate members being attached to one another. Such joining may be permanent in nature or alternatively may be removable or releasable in nature. Similarly, coupled can refer to a two member or elements being in communicatively coupled, wherein the two elements may be electronically, through various means, such as a metallic wire, wireless network, optical fiber, or other medium and methods.

As used herein, the terms “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element without departing from the teachings of the disclosure.

The IVPA imaging apparatus and method of the present disclosure is configured to provide a foundation for building a multimodal platform for imaging lipid-laden, vulnerable plaque due to its unique capabilities of chemically specific and depth-resolved detection of lipids. The IVPA imaging apparatus and method of the present disclosure may be used for: 1) characterizing the natural history and progression of vulnerable plaque; 2) identification of solitary vulnerable plaque to determine the efficacy of treatment interventions; 3) determination of the efficacy of preventative therapies (e.g. statins) to reduce lipid-core size. The multimodal IVPA-IVUS imaging apparatus and method of the present disclosure could open opportunities beyond the reach of other intravascular imaging tools.

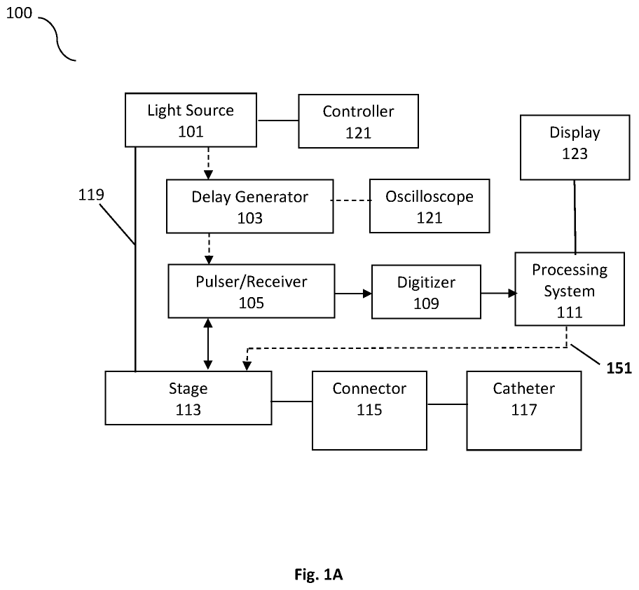

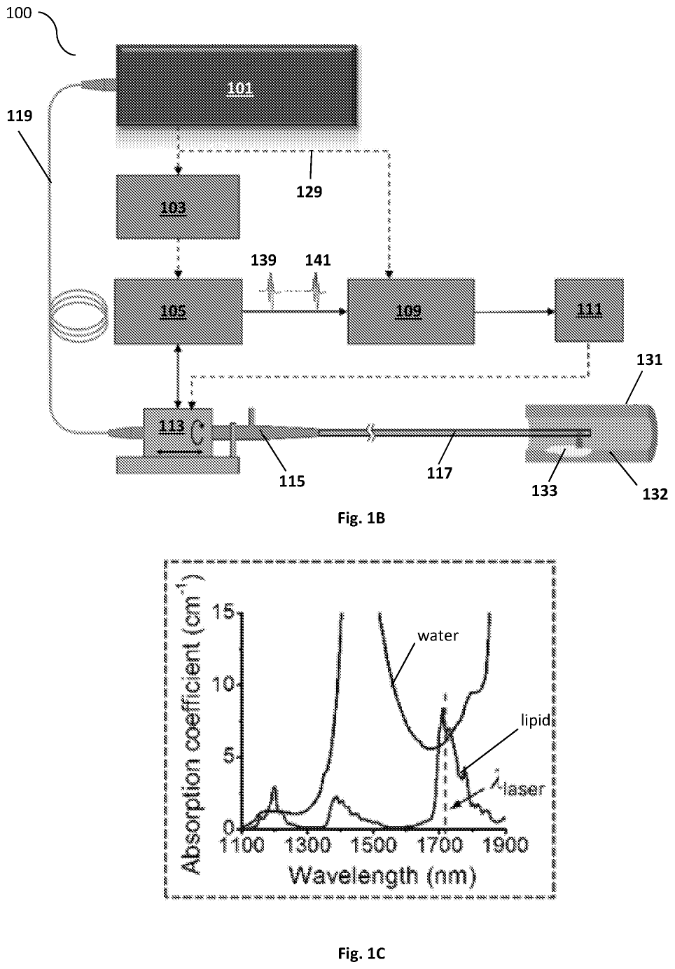

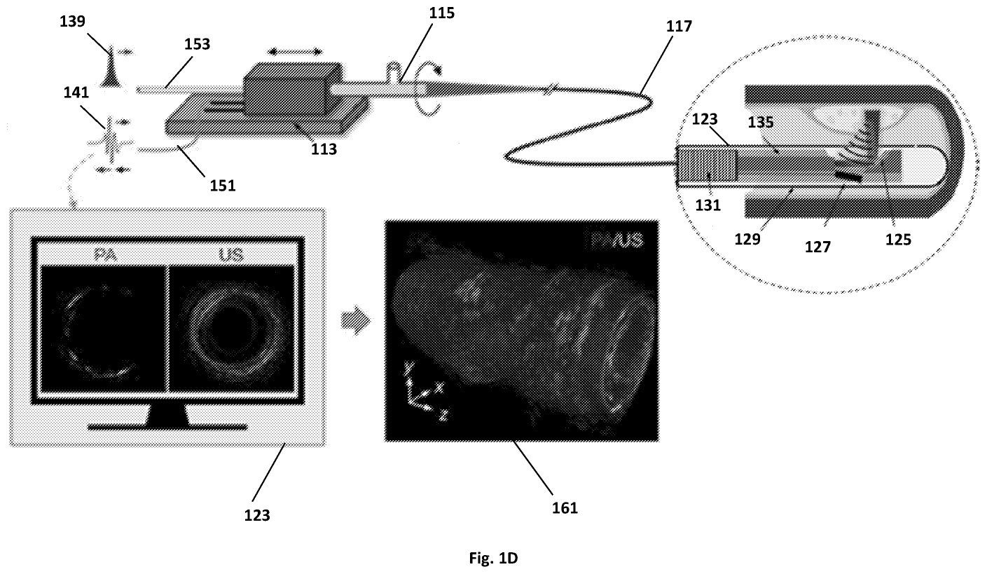

As shown in FIG. 1A, the present disclosure can include an imaging system 100 that can include various components, such as a light source 101, which can include a controller 121 to provider a user the ability to control the desired light source. The light source can be communicatively coupled to a delay generator 103 and/or a puller/receiver 105, which can further be communicatively coupled to a motorized stage 113. The stage 113 can be coupled to a connector 115 that is further coupled to a catheter 117. In some exemplary embodiments, the stage can be motorized and controlled using any suitable means. The stage 113 can be configured to move along one or more axis. The movement of the stage 113 can correspond to movement of the catheter, such as a pullback movement once, the catheter is placed into a pre-determined position. In one exemplary embodiment, a quasi-collinear IVPA catheter with high sensitivity and sufficient depth and selected a sheath material with minimal PA and US attenuation and artifact generation can be used. The advantages of exemplary embodiments of the quasi-collinear IVPA catheter of the present disclosure including enabling in vivo IVPA imaging of native arteries under clinically relevant conditions with real-time display up to 16 frames per second (fps), which were tested using a rabbit model. The imaging system allows for localization and quantification of lipid content, such as a plaque 133, to be performed along the full depth of the arterial wall from intima to perivascular adipose tissue for pullback lengths up to about 80 mm. The apparatus of the present disclosure can include a light source 101, a delay generator 103, a puller/receiver 105, a digitizer 109, processing means/computer 111, a coupling means 119, a connector 115, a stage 113, such as a hybrid rotary joint and motorized stage, and a catheter 117. In some exemplary embodiments, the connector can be a Luer Slip type connector, however, any suitable connector can be used. In one exemplary embodiment, the apparatus can include a chiller and oscilloscope.

In one exemplary embodiment, the apparatus can include a high-speed IVPA tomography system configured to provide dual-modality intravascular photoacoustic imaging and ultrasound imaging at speed up to 16 fps with real-time display 123. The processing means 111 can control various elements of the apparatus and coordinate the elements to operate the apparatus of the present disclosure. The light source 101 can be an excitation light source, such as a laser beam. In one exemplary embodiment can be a Nd:YAG pumped OPO (Nanjing Institute of Advanced Laser Technology) that can emit a beam at various pulses, repetition rates, and at a range of wavelengths, which can operate as the photoacoustic signal 139 and ultrasonic pulses 141. In some exemplary embodiments, the light source can emit a beam between the light source emits a pulse between 2-ns and 20-ns with between a 1 kHz and 5 kHz repetition rate at a wavelength between about 1600 nm and 1900 nm. In one exemplary embodiment, the light source can emit a beam at about a 10-ns pulse with about a 2-kHz repetition rate at a wavelength of about 1730 nm. The light source 101 can be coupled to the imaging catheter 117 using a coupling means 119, such as and in some exemplary embodiments can include a multimode fiber or optical fiber. The catheter 117 can then be directed to and positioned within the arterial wall of the artery 131 of a subject or patient for lipid-specific excitation. The catheter can have a first end 171 and a second end 173. The first end of the catheter can be coupled to the connector 115 which can be couple to the stage 113. The second end of the catheter 117 can include the imaging probe portion 123. The system can further include a motorized stage 113. In one exemplary embodiment, the stage 113 can be a hybrid optical and electrical rotary joint can be used for efficient optical coupling and radiofrequency signal transmission at fast rotation of the catheter or imaging prober. The stage 113 can be configured to rotate the connector 115. The system can use quasi-collinear IVPA catheter with an outer sheath can be used for intravascular PA/US imaging as illustrated in FIG. 1D. The output pulse energy from the imaging probe portion 123 of catheter tip can be controlled by the controller 121. Similarly, in some exemplary embodiments, the processing means can further act as the controller. The stage can allow for the movement of the catheter in both a rotational direction as well as along an axis for pullbacks from a patient.

In some applications, the output pulse energy is controlled to be around 100 μJ, corresponding to a laser fluence of about 50 mJ/cm², which is below the ANSI laser safety standard of about 1 J/cm² at 1730 nm. Additionally, the ultrasound pulses 141 can be delayed and triggered by a pulse generator 103 (Model 9512, Quantum Composers, Inc.) can sent/received by a puller/receiver 105 (5073PR, Olympus, Inc.) to provide co-registered ultrasound image of the artery 131. In some application this pulse can be delayed by about 5 μs. The ultrasonic signal 141 can be carried through a third coupling means 151, which in some embodiments can be an electrical wire to that is coupled to the connector 115. The connector can have a first end and a second end. In some embodiments, the connector 115 can include an electrical connector 155 and a fiber connector 157 on the first end, which can correspond to an electrical wire 151 and first coupling means 119 respectively. The second end can be coupled to a catheter 117, which can house the optical fiber 119 and electrical 151 as shown in FIG. 1D and 1E. The catheter can include a housing or sheath 129 to protect the internal imaging elements within the catheter 117. A processing means 111 can be used for control, processing, real-time display, and data collection. Additionally, the entire imaging system can be installed on a portable cart for easy movement. As shown in FIG. 1B and 1D, the coupling means 119, which can include an optical fiber 153, and can transmit the beam 139 from the light source 101 to the connector 115. Similarly, the ultrasonic signal can travel both to the connector 115 via an electrical wire 151 and can also travel back from the connector 115 after the artery has been imaged and travel to the processing means 111. The processing means can the take the imaging data and signals which can be used to generate a 3D reconstruction 161 to be displayed on the display 123 in real-time or stored on the processing means 111 which can include a memory.

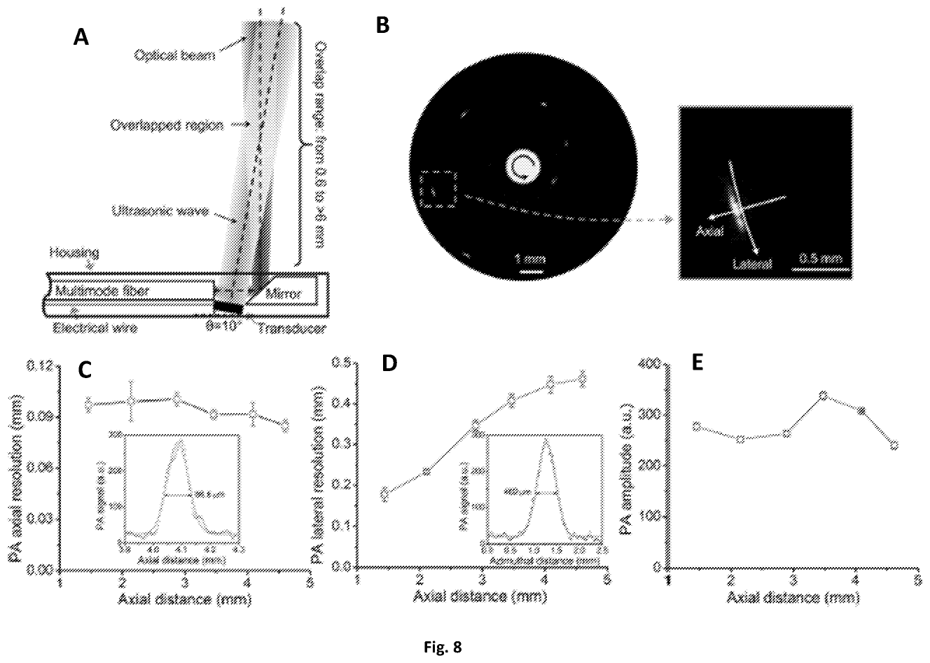

The apparatus can include a quasi-collinear IVPA catheter design configured for high sensitivity in vivo applications (FIG. 1D and FIG. 8A). Similar to the coupling means 119, a second coupling means can be used to deliver the light source to the imaging probe portion 123. In one exemplary embodiment, the second coupling means 135 can be a multimode fiber (FG365LEC, Thorlabs) that can be used for high-power laser pulse delivery. This second coupling means can be part of the first coupling means 119 that extends from the light source 119 or separate from the first coupling means 119. In one exemplary embodiment, the imaging probe portion 123 can include a mirror 125 or reflecting means, such as a rod or a fiber-end mirror polished to about 45° and coated with gold can be used for optical direction to the artery wall. A transducer 127, such as an US transducer (0.5×0.6×0.2 mm³, 42 MHz, 50% bandwidth) (AT23730, Blatek Industries, Inc.) can be used for PA detection and US pulsing/receiving. The transducer 127 can be positioned proximate to the rod mirror 125 and tilted at a desired angle. The transducer can be communicatively coupled to the processing means 111 to provide the signal received by the transducer for further processing. In one exemplary embodiment, the mirror 125 can be tilted about 10° forward to maximize the overlap between US and optical waves to realize a quasi-collinear PA detection, and to reduce the multiple US reflection from the protective sheath 129. In some embodiments, portions of the catheter can have different sheathing or housing compositions. The imaging probe portion can have a same or different housing or sheathing as the remainder of the catheter. This overlap region is further illustrated in FIG. 8A. The overlap depth can be estimated using the processing means. IN some embodiments, the overlap depth estimation can be from about 0.6 mm to about >6 mm by geometrical calculation considering the dimension of components and reasonable divergence angles of about 6° for optical beam and about 3° for US wave. The components can be positioned within a sheath or housing 129. In one exemplary embodiment, a 3D printed plastic housing (Proto Labs) can be used for the housing 129 and can be further protected by a stainless-steel tube. The catheter 117 rotation was transferred to the tip via a torque coil 131 or other suitable rotational coils. A sheath can be used to protect the entire imaging probe portion for in vivo application and specifically include properties to better image the interior of an artery.

In some exemplary embodiments, the diameter of the imaging catheter and sheath can be from about 2 mm to about 0.5 mm or about 1.6 mm to about 1.0 mm for safe coronary artery access. A thinner optical fiber and rod mirror, smaller diameter torque coil, better integration of catheter components, and thinner catheter sheath can be used to reduce the diameter of the imaging catheter. In some exemplary embodiment, the sheathing can house the imaging probe portion of the apparatus as shown in FIG. 1C-D.

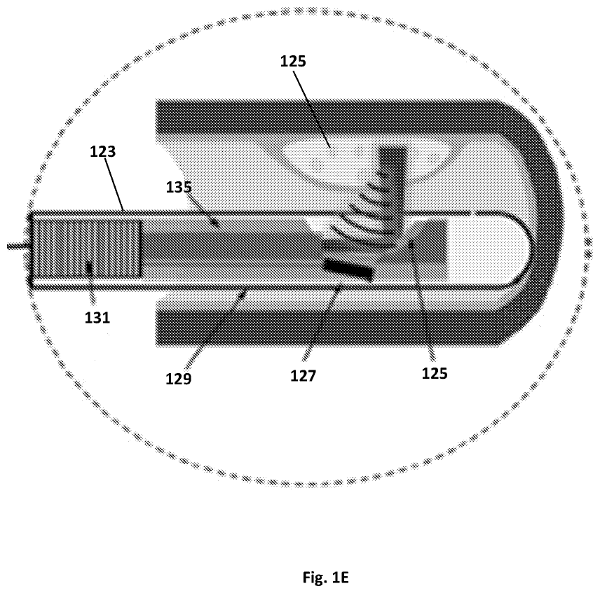

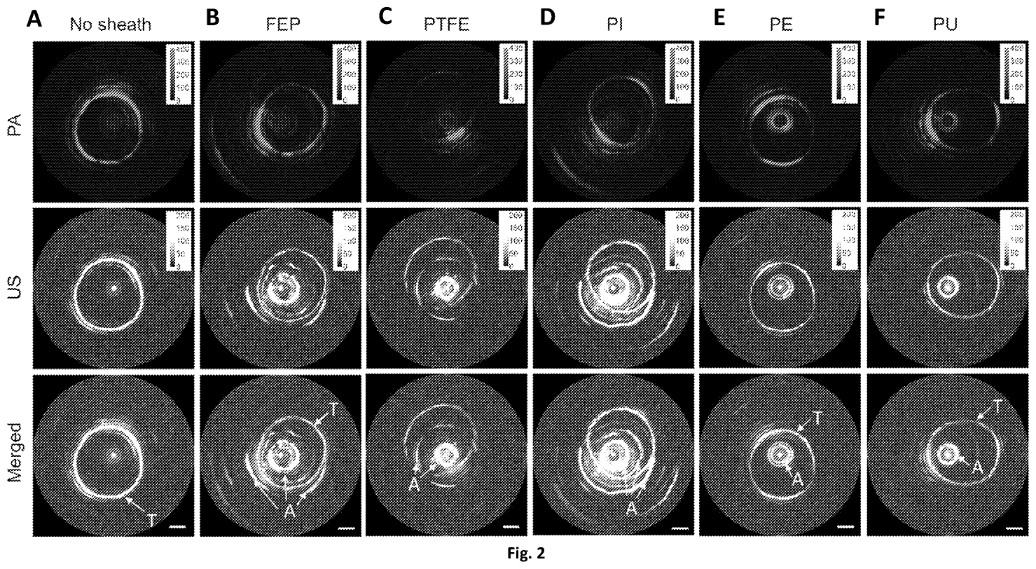

The sheath can be comprised of any suitable material. In order to find a proper sheath material, five different polymers were selected and tested as candidate based on their optical and acoustic properties, (i.e. low optical absorption at about 1.7 μm and matched acoustic impedance with aqueous medium below Table 1). To test their PA and US behavior, the polymers were fabricated into tubes with proper dimension to fit the IVPA catheter, and a heat-shrink tube was imaged with/without these sheath materials, as shown in FIG. 2. PA/US artifact generated from and transmission over the sheath were analyzed to provide criteria for sheath material selection.

The sheath material can be further optimized from other polymers to further improve the imaging quality by reducing the transmission losses and avoiding unnecessary artifacts from the sheath. A broadband transducer covering the low-frequency PA signal, typically in several MHz range, while maintaining US resolution needs to be developed for better imaging quality.

In addition, all materials used for catheter fabrication may adhere to regulatory control for biosafety to allow for clinical use of the imaging system of the present disclosure.

TABLE 1
Optical and acoustic properties of sheath material candidates and liquid media.

| | Optical properties | | | Acoustic properties | | | | |
|---|---|---|---|---|---|---|---|---|
| | | | μₛ | ρ | cₛ | Z | αₛ | |
| Material | n | μₐ (cm⁻¹) | (cm⁻¹) | (kg/m³) | (m/s) | (MPas/m) | (dB/cm) | Chemical Structure |
| Water | 1.31 | 7.41 | 0.07 | 1000 | 1450 | 1.45 | 3.472 | 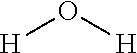 |
| Heavy water | 1.31 | 0.12 | 0.07 | 1000 | 1450 | 1.45 | 3.472 | 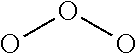 |
| PI | 1.7 | 2* | — | 1420 | 2246 | 3.19 | — | 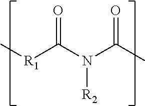 |
| PE (LD) | 1.5 | 10* | — | 920 | 2080 | 1.91 | — | 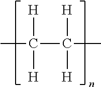 |
| PU | 1.5 | 4* | — | 1200 | 1900 | 2.28 | — | 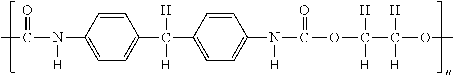 |
| FEP | 1.34 | 0.1* | — | 2150 | 1330 | 2.86 | — | 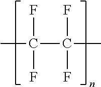 |
| PTFE | 1.34 | 0.1* | — | 2160 | 1400 | 3.02 | — | 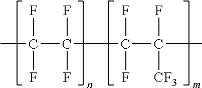 |

PI, Polyimide; PE, Polyethylene; LD, low density; PU, Polyurethane; FEP, Fluorinated ethylene propylene; PTFE, Polytetrafluoroethylene. n, refractive index; μₐ, absorption coefficient; μₛ, scattering coefficient; ρ, density; cₛ, speed of sound; Z, acoustic impedance; αₛ, acoustic loss. The optical properties correspond to optical wavelength of 1.7 μm and acoustic loss is for a frequency of 40 MHz. *estimated from their chemical structure and photoacoustic signals.

### EXPERIMENTAL

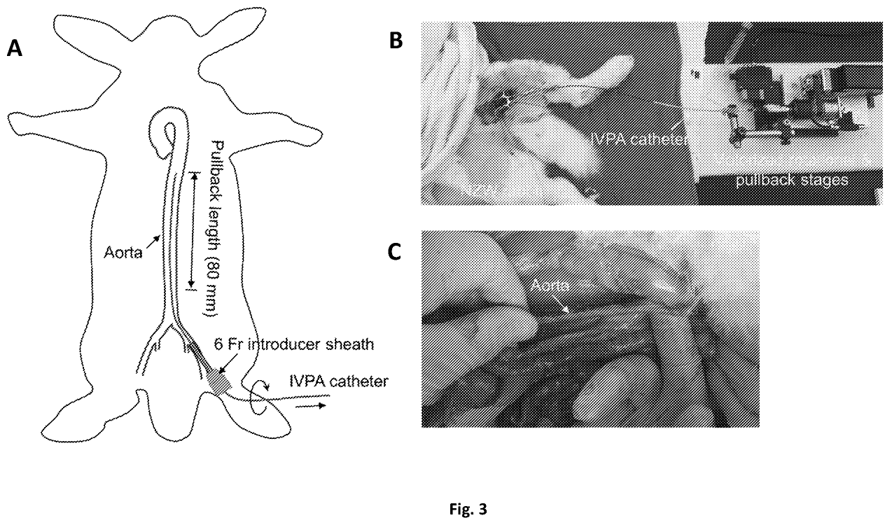

Testing of the apparatus and method of the present disclosure was performed according to the Animal Studies for Cardiovascular and Intestinal Imaging and approved by the Purdue Animal Care and Use Committee. Three male New Zealand White (NZW) rabbits (Charles River Laboratories), aged eight months old and fed with a normal chow diet, were used for in vivo IVPA imaging. Before imaging procedure, the rabbit was anesthetized with a proper dose of ketamine (about 35 mg/kg) and xylazine (about 5-10 mg/kg) through ear vein injection and maintained on about 1-5% isoflurane mixed with about 100% O₂ via endotracheal intubation during the entire imaging process. A cutdown procedure was used to identify the left femoral artery for intravascular access. A 6 Fr introducer sheath was inserted in the femoral artery, through which the IVPA catheter was advanced to the thoracic aorta (FIG. 3A, B), guided by x-ray angiography. The catheter sheath was flushed with D₂O to reduce optical loss and remove laser heating during IVPA imaging. Different rotational and pullback speed combinations (4 fps and 0.25 mm/s, 16 fps and 1 mm/s) were used to confirm the reproducibility of our imaging system. A total length of 80 mm was recorded for each pullback. Following imaging, the rabbit was euthanized by using intravenous euthanasia solution (390 mg/ml) and the aorta was harvested for histology (FIG. 3V).

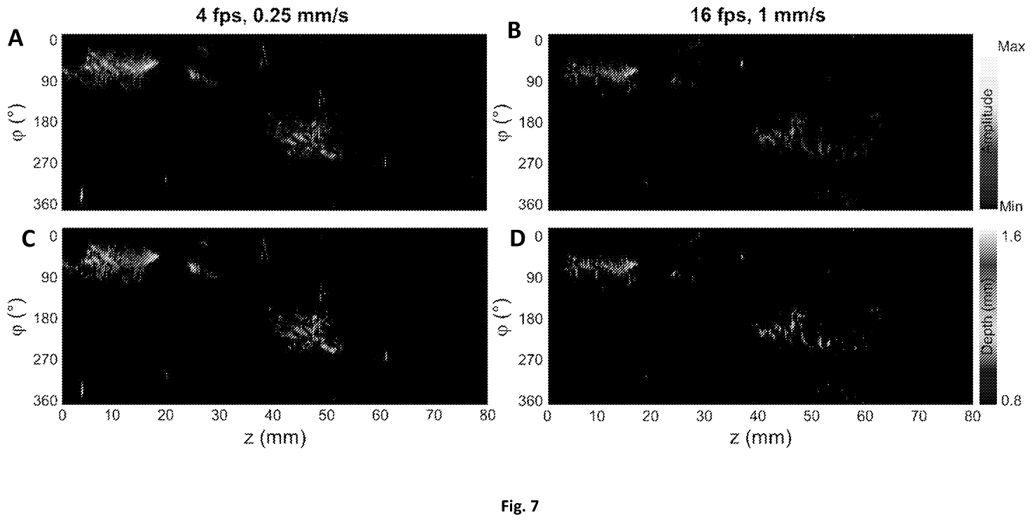

Human tissue samples were tested and approved by Human Research Protection Program of Purdue University and performed in accordance with the approved guidelines. The informed consent was obtained from all subjects. A fresh, human heart was harvested from a 44-year-old female undergoing transplant surgery within 24 hours. Immediately, the coronary arteries were excised and cannulated with a 6 Fr introducer sheath, sutured in place (FIG. 7A). The artery was then pinned flat into a container and submerged in 1×PBS. The IVPA catheter with sheath was advanced distally, approximately 40 mm past the introducer sheath. During imaging, the artery was perfused with 1×PBS at room-temperature and catheter was flushed with D₂O. Pullback was recorded at 16 fps and 0.5 mm/s for a total length of 40 mm.

All arteries were pressure fixed in 10% w/v formalin at approximately 25 mL/min for about 30 minutes to maintain lumen as close to in vivo morphology as possible. The arteries were then grossly sectioned in 3-4 mm segments and paraffin embedded, sectioned, and stained for Verhoeff-van Gieson and Russel-Movat's pentachrome.

The apparatus and method of the present disclosure uses IVPA tomography hybrid intravascular imaging technology having both optical absorption-based contrast for depth-resolved lipid-specific mapping and traditional ultrasound detection for deep tissue morphology (FIG. 1C). Currently, sensitivity remains the most important technical challenge for IVPA to be applied to in vivo study. To address this problem, a quasi-collinear catheter was constructed and used (see Methods, FIG. 1D and FIG. 8A), the diameter of which including outer sheath was measured to be about 1.6 mm at the tip (FIG. 1D), and integrated it with our high-speed IVPA imaging system (FIG. 1A-B). The spatial resolution and imaging depth of the catheter with protective sheath was evaluated by imaging a 7-μm carbon fiber placed at different distances from the probe as shown in FIG. 8b. To maintain a detectable PA signal for a small target, the experiments were performed in deuterium oxide (D₂O) to reduce optical attenuation in the medium. The axial resolutions are measured to range from 85 to 100 μm, while the lateral resolutions are found to increase from 170 to 450 μm with increased depth, attributed to the divergence of the US propagation (FIG. 8C, D). The PA amplitude, affected by both the light intensity and optical beam and ultrasonic wave overlap, was detected within a depth range from 1.4 to 4.6 mm (FIG. 8E), sufficient to image the entire arterial wall.

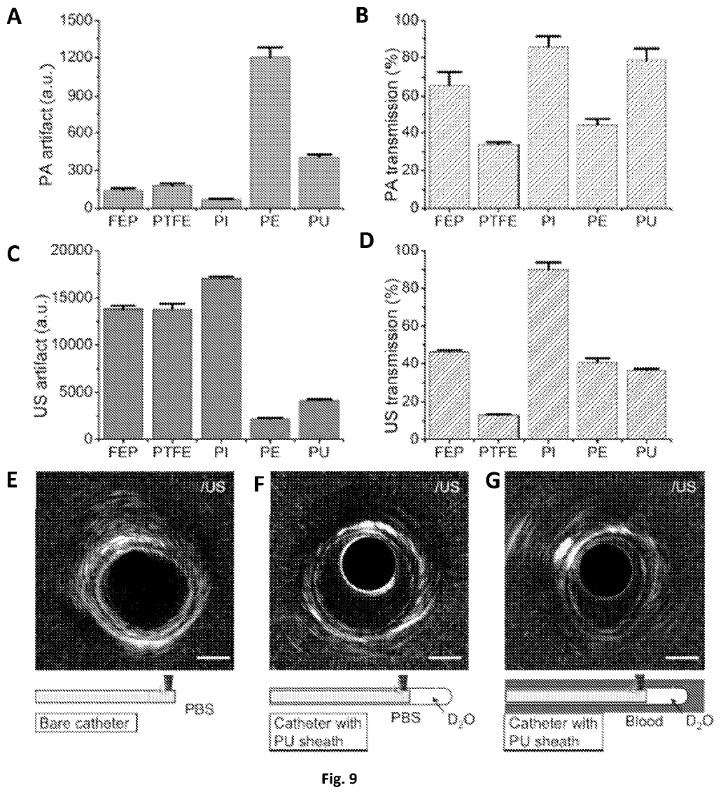

A sheath for IVPA catheter can be used to provide necessary protection to endothelia from damage by fast-rotating catheter as well as to the catheter from mechanical damage due to blood, thrombus, or the catheterization procedure. A functional IVPA sheath material should be optically and acoustically transparent, to reduce attenuation of PA and US signals to a minimum and induce minimal artifacts. Proper sheath material can be selected based on their optical and acoustic properties (Table 1). Some of these materials were tested for performance by imaging a heat-shrink tube with our quasi-collinear catheter. The imaging results are shown in FIG. 2 with comparison with a bare catheter. Their performance in term of induced artifacts and transmission for PA and US signals was also summarized in FIG. 9A-D. Although fluorinated ethylene propylene, polytetrafluoroethylene, and polyimide induced minimal artifacts for PA images, their overwhelming US artifacts make them difficult to be selected as proper sheath materials (FIG. 2B-D). Compared with polyethylene, polyurethane (PU) exhibits a smaller PA artifact, a larger PA transmission and comparable US behavior (FIG. 2E, F and FIG. 9A-D), thus was selected as our material of choice for the sheath in imaging window section (FIG. 1D).

The PU sheath with dimension adapted to the imaging catheter was further evaluated by ex vivo imaging of a human coronary artery in different environments (FIG. 9E-G). The catheter with a D₂O-filled PU sheath demonstrated comparable or even stronger PA intensity and moderate US attenuation as compared to imaging with the bare catheter in phosphate buffered saline (PBS) (FIG. 9E, F). In other words, the optical loss across the sheath material was compensated by filling the sheath with D₂O, which has a much smaller absorption coefficient than water at 1.7 μm. Furthermore, IVPA imaging with PU sheath in the presence of luminal blood (FIG. 9g) demonstrated the capability of our imaging system for in vivo intravascular imaging without luminal blood flushing or occlusion, which is an important advantage over other optical imaging modalities such as optical coherence tomography in clinical applications. The following in vivo imaging experiments were based on the scheme described in FIG. 9G.

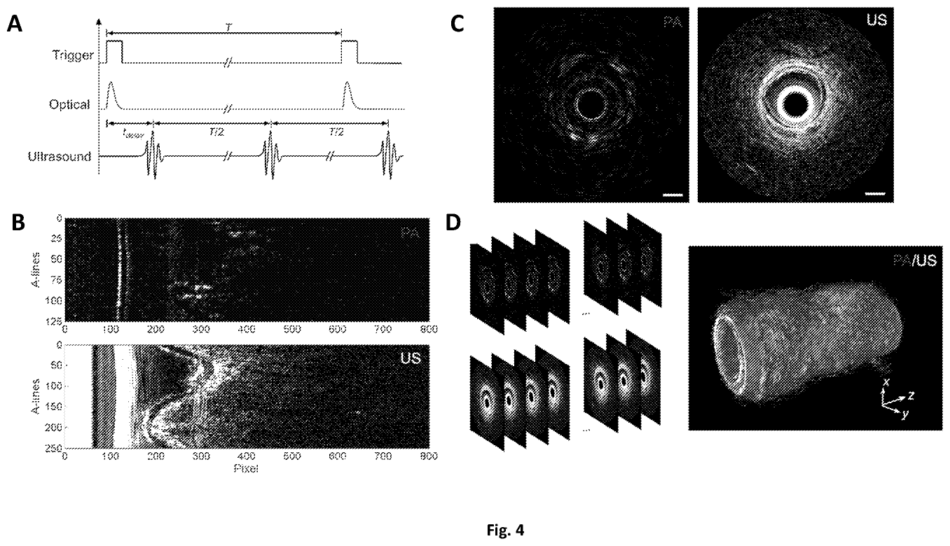

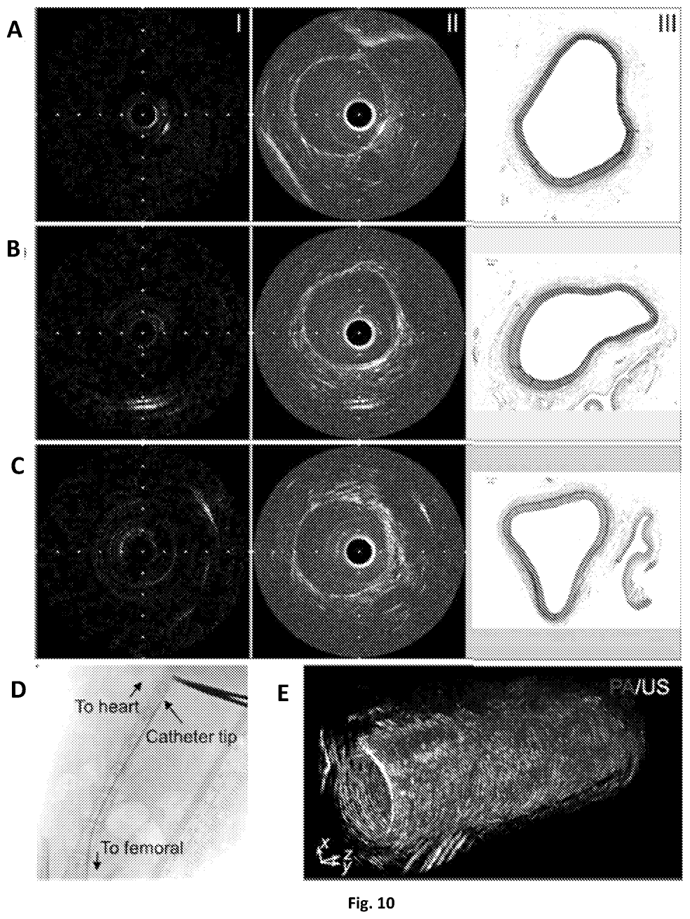

The in vivo performance of the imaging apparatus and method of the present disclosure was tested by the thoracic aorta of three lean, male NZW rabbits. The catheter was placed through femoral artery under x-ray angiography (FIG. 10D). In vivo IVPA images of the aorta with about 80-mm pullbacks were recorded at different rotational and pullback speeds, up to about 16 fps and about 1 mm/s (Supplementary Videos S1 and S2), respectively. FIG. 10A-C shows representative cross-sectional PA (I), merged PA/US images (II), and histology results (III) at different positions corresponding to the distal, upper and proximal sections of thoracic aorta (FIG. 3A). The PA images show the presence of lipid within the aorta wall (FIG. 10A) and perivascularly at depths greater than about 4 mm (FIG. 10B, C). The US images provide important morphological information about the artery, such as luminal area and thickness of artery wall. Given the young age and lean diet of the NZW rabbits, and the histology did not show any vascular pathology. The abundance of perivascular adipose tissue agrees with the strong PA signals detected peripherally in the corresponding sections (FIG. 10B, C). Reconstructed 3-dimensional (3D) PA/US merged image with about a 20-mm pullback length (FIG. 10E and FIG. 4) illustrates the detection and presence of perivascular adipose tissue at the proximal end of the pullback, close to the femoral artery.

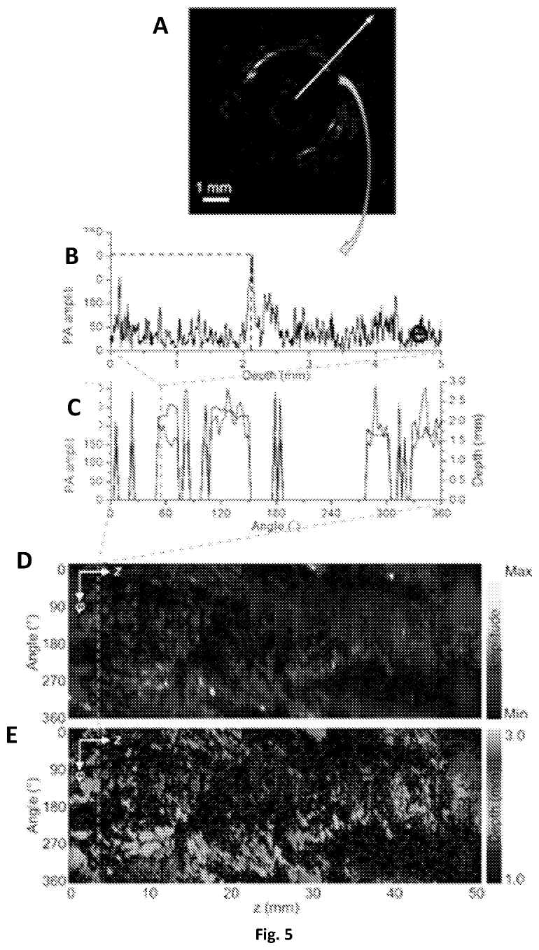

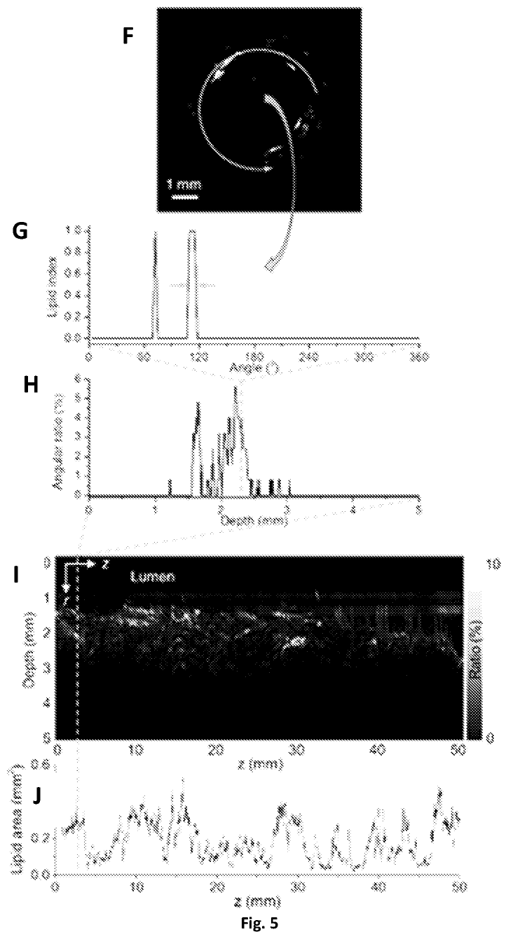

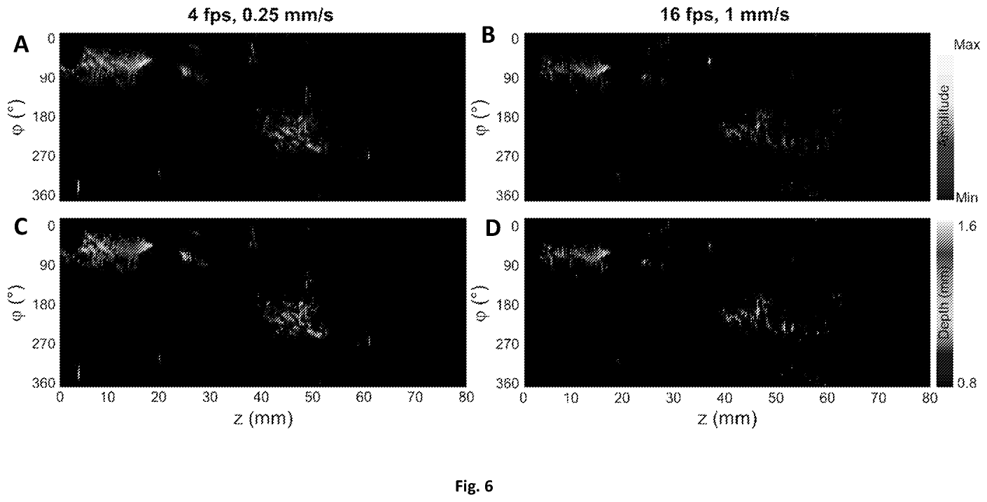

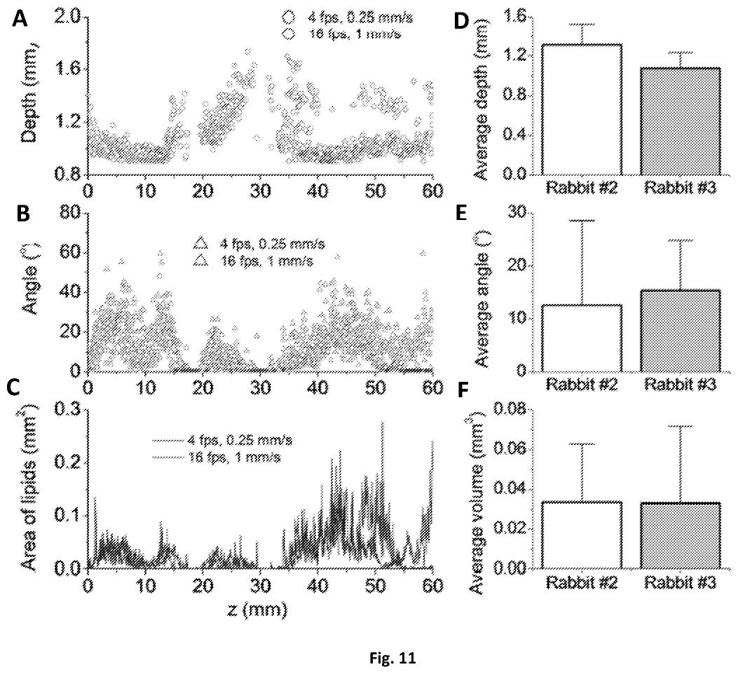

Imaging performance was compared by imaging the thoracic aorta of another rabbit in terms of lipid core depth, observation angle and lipid area (FIG. 5) at different rotational and pullback speeds (4 fps and 0.25 mm/s vs. 16 fps and 1 mm/s). Similar results were observed (FIG. 11A-C and Supplementary FIG. 6), confirming the reproducibility of our imaging system and protocol. The averaged results for two rabbits along 60-mm pullbacks further confirmed the healthy aorta of the rabbits on lean diet (FIG. 11D-F).

As shown in FIGS. 5A-J, cross-sectional PA images were reconstructed. The cross-sectional photoacoustic image was reconstructed from raw data obtained (FIG. 5A). The maximum PA intensity along the radial direction and its corresponding depth from the catheter center were calculated for each frame (FIG. 5B, C) to generate two-dimensional maps of lipid presence and depth (FIG. 5D, E), which provides an overview of depth-resolved lipid distribution. A binary lipid index image (i.e. 0 for background and 1 for lipid) was generated by applying a well-chosen threshold (4 times of background noise in this work) to the PA images (FIG. 5F). The threshold was determined from a series of integrals that corresponds to optimal match between PA images and lipid index images. The angular ratio of biggest lipid pool at each depth, i.e. angle of field of view over 2π, was generated for every frame (FIG. 5G, H) and plotted for the entire pullback length (FIG. 5I) to give complementary information about the lipid-core size and depth. The lipid area in each frame was calculated based on the binary lipid index image and plotted against the pullback length to visualize the total lipid deposition longitudinally (FIG. 5J).

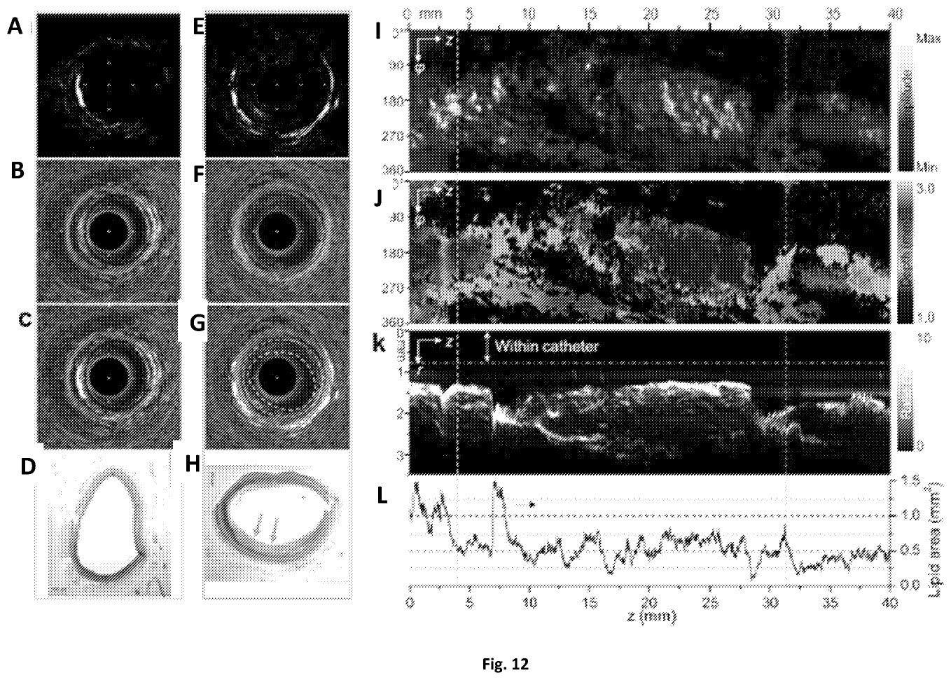

For ex vivo testing performance and validation, the apparatus and method of the present invention was used for the detection of true coronary pathology and future translational applications, the imaging system was further tested on a human right coronary artery ex vivo. The IVPA catheter with sheath was advanced about 40 mm into the distal artery and imaged at about 16 fps and pullback speed of about 0.5 mm/s with constant perfusion with PBS. Results are shown as cross-sectional photoacoustic (FIG. 12A, E), ultrasound (FIG. 12B, F) and merged PA/US (FIG. 12C, G) images. Corresponding histopathology result (FIG. 12D, H) with Movat's pentachrome stain at representative locations was also displayed for confirmation. A short movie composed of merged PA/US images and their pullback view was provided in Supplementary Video S3. Strong photoacoustic signals were observed outside the vessel from perivascular adipose tissue, while obvious photoacoustic signal was detected from the thickened intima layer (7 o'clock) as well (FIG. 12E-G), which is very likely from lipid-rich plaque as highlighted by color outlines in FIG. 12g and confirmed by histology result in FIG. 12H (arrows). Additionally, ex vivo angiography with contrast shows a small lesion (indicated by arrowhead) approximately 10 mm from the introducer sheath (indicated by arrow), corresponding to the thickened region in the histology section shown in FIG. 12h (arrows). The 2-dimensional lipid distribution and depth maps at the peaks of photoacoustic A-lines are shown for a 40-mm segment of the artery (FIG. 12I, J). Dense lipid distribution along the entire pullback was observed with a depth ranging from about 1 mm to about 3 mm. Angular ratio of the maximum lipid pools, i.e. the angle of view over 2π in percentage, at each individual depth was calculated frame by frame for the entire pullback (FIG. 12K and FIG. 5), which further helps to quantify the lipid core size and depth in lipid-rich plaque identification. The total lipid area was quantitated for each cross-section along the artery (FIG. 5) and presented with alignment to lipid distribution maps (FIG. 12I-K) to show the variation of lipid accumulation within and outside the vessel wall (FIG. 12L). The reconstructed 3D images in different views (FIG. 7) illustrate lipid distribution pattern in relation to the artery morphology.

While the invention has been described above in terms of specific embodiments, it is to be understood that the invention is not limited to these disclosed embodiments. Upon reading the teachings of this disclosure many modifications and other embodiments of the invention will come to mind of those skilled in the art to which this invention pertains, and which are intended to be and are covered by both this disclosure and the appended claims. It is indeed intended that the scope of the invention should be determined by proper interpretation and construction of the appended claims and their legal equivalents, as understood by those of skill in the art relying upon the disclosure in this specification and the attached drawings.

What is claimed is:

1. An intravascular photoacoustic tomography apparatus, comprising:
a light source configured to emit a light beam at a desired wavelength;
a pulser receiver configured to send and receiver the ultrasound pulses;
a delay generator configured to delay and trigger the ultrasound pulses;
a processing means configured to control the pulser receiver, light source, and delay generator;
a connector having a first end and second end;
a coupling means configured to communicatively couple the light source to the first end of the connector;
a catheter having a first end and a second end, wherein the catheter is coupled to the second end of the connector, wherein said catheter comprises an imaging probe portion, wherein said imaging probe portion comprises a mirror, transducer, and optical fiber; and
a stage coupled to the first end of the connector, wherein the stage is configured to move along at least a first axis.

2. The intravascular photoacoustic tomography apparatus, wherein said stage is a motorized stage comprising a hybrid rotary joint and motorized portion, wherein said motorized portion is configured to move along the first axis to linearly pullback the catheter and the rotary joint is configured to rotate around a second axis.

3. The apparatus of claim 2, wherein said light source is a laser is an optical parametric oscillator (OPO) pumped by the second harmonic of a Nd:YAG.

4. The apparatus of claim 3, wherein the delay generator provides delayed ultrasound pluses sent by a puller receiver to provide co-registered ultrasound images.

5. The apparatus of claim 4, wherein the pulses are delayed by 5 μs.

6. The apparatus of claim 4, wherein the light source emits a pulse between 2-ns and 20-ns with between a 1 kHz and 5 kHz repetition rate at a wavelength between about 1600 nm and 1900 nm.

7. The apparatus of claim 5, wherein the light source emits a pulse 10-ns with 2-kHz repetition rate at a wavelength of about 1730 nm.

8. The apparatus of claim 4, wherein the light source is coupled to the imaging catheter via a multimode fiber, wherein the multimode fiber is an optical fiber.

9. The apparatus of claim 8, wherein the fiber and catheter are coupled using a hybrid optical and electrical rotary joint configured to provide efficient optical coupling and radiofrequency signal transmission at a fast rotation.

10. The apparatus of claim 9, wherein the catheter comprises an outer sheath.

11. The apparatus of claim 10, wherein imaging probe portion further comprises a torque coil.

12. The apparatus of claim 11, wherein the outer sheath is composed of polyurethane.

13. The apparatus of claim 12, wherein the outer sheath is filled with a D₂O solution prior to imaging the interior of an artery.

14. The apparatus of claim 8, wherein the catheter is configured to be inserted inside an artery having an artery wall for detecting lipid deposits.

15. The apparatus of claim 14, wherein the light beam is directed from the light source by the optical fiber to the imaging probe portion of the catheter, wherein the light beam is reflected by the mirror towards a focus spot of interior tissue of the artery wall for photoacoustic stimulation by the light source.

16. The apparatus of claim 15, wherein the mirror is positioned 10° forward to maximize the overlap between the ultrasonic signal and optical waves and reduce ultrasonic signal interference.

17. The apparatus of claim 16, wherein the transducer is configured to capture ultrasonic signals generated from interior tissue of the artery wall.

18. The apparatus of claim 17, wherein the stage is configured to move from a first position to a second position along the first axis to reposition the focus spot of interior tissue.

19. The apparatus of claim 17, further comprising a display, wherein the processing means is configured to use the ultrasonic and signals to generate a three-dimensional reconstruction image of the interior tissue of the artery wall.

20. A method for imaging an artery wall for lipid deposits, comprising:
providing an IVPA apparatus comprising a light source, a delay generator, a puller/receiver, a digitizer, processing means/computer, a multimode fiber, a Luer-slip connector, a hybrid rotary join and motorized stage and a catheter;
inserting the catheter inside an artery having an artery wall,
directing the light source via the multimode fiber and catheter to the arterial wall for lipid-specific excitation;
photoacoustically stimulating said artery wall with optical energy via an imaging probe with the light source and multimode fiber;
capturing the ultrasonic signals generated from said tissue via the transducer array, and
repositioning focus spot of the artery wall by pulling back the catheter through the artery a pre-determined distance and direction and repeating the steps of stimulating the artery wall and capturing ultrasonic signals; and
generating an image of the tissue by combining the captured photoacoustic and ultrasonic signals from the first scanning.
